(19) United States
(12) Patent Application Publication
(10) Pub. No.: US 2007/0059268 A1
(43) Pub. Date: Mar. 15, 2007

(54) COMPOSITIONS, METHODS AND KITS FOR TREATING ALLERGIC DERMATITIS OF SKIN

(76) Inventor: Laura Magee, Milltown, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/464,986

(22) Filed: Aug. 16, 2006

Related U.S. Application Data
(60) Provisional application No. 60/715,647, filed on Sep. 9, 2005.

(51) Int. Cl. A61K 8/46; A61K 8/31

(52) U.S. Cl. 424/70.21; 424/70.31

(57) ABSTRACT

The invention includes compositions suitable for removal of urushiol from skin having been exposed thereto, which compositions contain an ethoxylate and sodium lauroyl sarcosinate, methods for treating allergic dermatitis of skin having been exposed to urushiol, and kits suitable for treatment of allergic dermatitis,

### FIELD OF THE INVENTION

The present invention relates to treatments for allergic dermatitis, e.g.

Toxicodendron dermatitis resulting from contact with the Rhus oleoresin urushiol, and to compositions suitable for use in such treatments.

### BACKGROUND OF THE INVENTION

Urushiol is the toxin responsible for the allergic dermatitis caused by contact with the sap of commonly encountered noxious plants such as poison ivy, poison oak, poison sumac, and related plants found throughout the world. Chemically, urushiols are mixtures of catechols with long, hydrophobic, carbon(alkyl) side chains at the three position of the catechol ring. For example, poison ivy contains predominantly 3-n pentadecylcatechols (C-15) and poison oak contains predominantly 3-n-heptadecylcatechols (C-17).

When skin is exposed to the oleoresin containing the urushiol, the reaction is an allergic eczematous contact dermatitis characterized by redness, swelling, papules, vesicles, bullac and streaking. Treatment of the reaction has historically consisted of attempting to remove the oil as quickly after exposure as possible. If the oil is not removed within a short period of time of exposure, e.g. a matter of a few minutes, attempts to remove the toxin thereafter may not be successful in preventing the allergic reaction, although it may limit its spread.

Attempts have been made to find both prophylactic treatments as well as post-exposure treatments. For example, treatment for poison ivy using a composition at includes linseed oil, an astringent,.a starch, an essential oil and citrus oil are known. However, linseed oil can cause irritation to the skin itself In addition, the composition must be applied to the effected areas up to twice a day until the rash is gone.

Other known treatment for dermatitis caused by exposure to the toxin urushiol includes application of compositions that contain a mixture of nonyl phenyl ethoxylates, followed by removal of the composition from the skin to remove urushiol. However, there are environmental concerns due to the presence of nonylphenols. In addition, in such methods of treatment, the composition is removed within a relatively short period of time and may require multiple applications of the composition. In addition, such treatments leave no therapeutically effective active ingredients on the skin to continue treatment of the allergic reaction.

It would be advantageous to provide a composition that is suitable for the removal of urushiol from the skin and that is more environmentally friendly. It also would be advantageous to develop a method of treatment of allergic dermatitis that would include not only removal of urushiol from the skin, but that also provides continued treatment after the cleansing composition is removed. The present invention provides environmentally acceptable compositions for the removal of urushiol from skin and methods and kits for use in the treatment of allergic dermatitis caused by exposure to urushiol.

### SUMMARY OF THE INVENTION

The present invention relates to compositions that are essentially free of nonyl phenyl ethoxylates and that are suitable for removal of urushiol from an area of skin having been exposed to urushiol. The compositions include water, an alcohol ethoxylate and sodium lauryl sarcosinate. The alcohol ethoxylate and the sodium lauryl sarcosinate are present in the composition in relative amounts effective to remove urushiol from the affected skin area exposed to the urushiol. The inventions also relate to kits including both a first composition suitable for removal of urushiol from the skin and a second composition suitable for treatment of allergic dermatitis, and to methods of treatment including use of the first and second compositions to remove at least a portion of urushiol from the affected area of skin and to provide continuing treatment of the affected area of skin after removal of the first composition and the portion of urushiol.

### DETAILED DESCRIPTION OF THE INVENTION

According to methods of the present invention for treating allergic dermatitis in an area of skin having been exposed to urushiol, an area of skin having been exposed to urushiol is contacted with a first composition comprising, in addition to other optional ingredients, an ethoxylate surfactant, sodium lauryl sarcosinate (SLS) surfactant and a suitable carrier for the surfactants. The ethoxylate and SLS surfactants are present in the composition at concentrations and in relative amounts effective to remove the urushiol from the affected area of skin.

The composition may be applied topically and may be delivered by any means know to those skilled in the art for applying topical medicaments, such as lotions, sprays, gels, or articles such as wipes or pads. The composition may be worked over the affected area in a scrubbing motion. The composition remains in contact with the skin area for a period of time effective to remove at least a first portion of the urushiol from the affected area of skin. The time period may range from a few minutes, e.g. 3 minutes, to an hour, or longer, although it is preferred to remove the composition as soon as possible to avoid prolonged exposure of the skin to the urushiol. After sufficient time has elapsed to ensure that the effected skin area has been adequately exposed to the composition, at least a portion of the composition and bound urushiol may be removed, for example by washing the skin.

While compete removal of the composition and bound urushiol is desired, depending on the amount of time and extent of scrubbing one spends in cleaning the effected area of skin, it is probable that in many cases, if not most cases, not all of the urushiol will be removed from the affected area of skin by cleansing with the first composition. Therefore, according to methods of the present invention, the affected area of skin is further contacted with a second composition comprising a therapeutically effective amount of an active ingredient suitable for treating allergic contact dermatitis caused by the urushiol. The second composition is applied topically and may be delivered by any means know to those skilled in the art for applying topical medicaments, such as lotions, sprays or gels. The second composition may remain on the effected area for extended periods of time, for instance 1 to 24 hours. The second composition may be applied more than once in order to provide sustained treatment of the effected area.

In certain embodiments of the invention, the ethoxylate may be selected from the group consisting of a nonyl phenyl ethoxylate and an alcohol ethoxylate, and may comprise mixtures of nonyl phenyl and alcohol ethoxylates. The composition may comprise two or more nonyl phenyl ethoxylates. The composition may comprise from about 3 to about 40 weight percent of the ethyoxylate and from about 2 to about 20 weight percent of sodium lauryl sarcosinate, based on the total weight of the composition. The ratio of ethoxylate to SLS used may vary depending on the particular ethoxylate used. The weight ratio of ethoxylate:SLS typically may range from about 1:2.5 to about 2.5:1.

In certain embodiments, the composition will be essentially free of nonyl phenyl ethoxylates. By essentially free, it is meant that the composition does not contain a nonyl phenyl ethoxylate at a concentration whereby it aids in the removal of urushiol or otherwise provides surfactant functionality to the composition. Such compositions will comprise less than 10 percent by weight of the nonyl phenyl ethyoxylate, preferably less than about 5 percent nonyl phenyl ethoxylates, more preferably less than 1 percent. Even more preferably, the composition will be free of nonyl phenyl ethoxylates. Such composition may comprise from about 3 to about 10 weight percent of the alcohol ethoxylate, and from about 2 to about 15 weight percent of SLS. Alcohol ethoxylates may include C₉-C₁₅, alcohol ethoxylates having HLB of from about 9 to 11. One alcohol ethoxylate used is Brij 30, a C₁₂ alcohol ethoxylate having a HLB of about 10, available from Uniqema, also know as Laureth-4 under CTFA/INCI nomenclature.

The compositions also may comprise an abrasive material to enhance removal of urushiol from exposed skin. The abrasive material assists in the physical detachment of urushiol from the skin and places it in position to more readily bond with the ethoxylate and SLS components of the composition. The beads should be large enough to be effective but not so large as to cause abrasions. In certain embodiments, beads in the range of 5 to 50 microns with an average size being approximately 25 microns, or 50 mesh, may be used. Compositions may comprise from about 5 to about 20 weight percent of the abrasive. Abrasives that may be used include polyethylene beads such as Inducos 13/4, available from InduChem, and microcrystalline wax beads such as Metabeads Microwax Green 28/60, available from Floratech. One skilled in the art will recognize that alternative abrasive materials may be used in the present invention.

Compositions of the present invention may further comprise thickeners, chelating agents, preservatives, emollients, opacifying agents, pH buffers and colorants, each in amounts effective to provide their respective functional property. The thickener may comprise an alkyl acrylate polymer, for instance Carbopol ETD 2020, available from Noveon. The chelating agent may comprise tetrasodium EDTA. Preservative may include methylparaben and phenoxyethanol. The balance of the composition may comprise water.

The second composition used in methods of the present invention may be aqueous based or non-aqueous based. In one composition, an external analgesic, .e.g. pramoxine HCL a skin conditioning agent, eg. Di-PPG-2 Myreth-10 Adipate, an astringent, e.g. zinc acetate and a denaturant, e.g. t-butyl alcohol and denatonium benzoate, are combined with ethylene alcohol and a film former, e.g. isobutylene/ethylmaleimide/hydroxyethylmaleimide copolymer in water and alcohol, to provide a composition suitable for treatment of allergic dermatitis caused by urushiol. The composition may comprise about 78 weight percent ethyl alcohol, about 20 weight percent film former (comprising about 8% copolymer, about 5% water and about 7% alcohol), about 1 weight percent external analgesic, about 0.75 weight percent skin conditioning agent, about 0.12 weight percent astringent, and about 0.1 weight percent denaturant, each based on the total weight of the composition. Other compositions including active ingredients suitable for treating allergic dermatitis also may be utilized.

The present invention also includes a kit suitable for treatment of allergic dermatitis in an area of skin having been exposed to urushiol. The kit comprises a first composition comprising an ethoxylate and sodium lauryl sarcosinate, as described herein above, in relative amounts effective to remove urushiol from an area of skin having been exposed to the urushiol; and a second composition comprising a therapeutically effective amount of an active ingredient suitable for treating allergic dermatitis, as described herein above. The kit may contain the first and second compositions within a single packaging element, or may include the first and second compositions in separate packaging elements. The first and second compositions in said kits may comprise lotions, gels or sprays. The kits also may include articles for applying the first and second compositions to skin, such as wipes containing either of the compositions. In particular, such wipes may include incorporated therewith amounts of the first composition that are effective to remove urushiol from skin, such that additional scrubbing of skin may be accomplished by the use of such wipes, thus providing removal of urushiol from skin. The compositions may be incorporated with the wipes by, for example, spraying or dipping.

The following examples are provided as examples of compositions of the present invention and/or useful in the methods and kits of the present invention and are not intended to limit in any way the scope of the claims appended hereto.

EXAMPLE 1

| Ingredient | Formula % (w/w) |
|---|---|
| Water | 78.6 |
| Glycerin | 2.0 |
| Sodium Lauroyl Sarcosinate | 11.7 |
| Laureth-4 | 5.0 |
| Tetrasodium EDTA | 0.5 |
| DMDMH | 0.9 |
| Methylparaben | 0.25 |
| Active organics | 0.1 |
| Citric Acid (20% sol.) | 0.68 |

A standard wipe was placed in an unsealed packet and the composition of Example 1 was applied to the standard wipe contained within the packet. The packet was then sealed for subsequent use. The composition may be applied to any wipes pad or other applicator suitable for use in the described application.

EXAMPLE 2

| | |
|---|---|
| Acrylates/C₁₀₋₃₀ Alkyl Acrylate Crosspolymer | 0.85 |
| Glycol Distearate | 1.25 |
| Coco-Glucoside | 0.5 |
| Glyceryl oleate | 0.15 |
| Glycerol stearate | 0.15 |
| Benzoic acid | 0.025 |
| Laureth-4 | 5.0 |
| Sodium Lauroyl Sarcosinate | 2.0 |
| Dowicil | 0.05 |
| Tetrasodium EDTA | 0.19 |
| Sodium hydroxide | 0.0005 |
| Polyethylene | 10.0 |
| Microcrystalline wax | 0.19 |
| Chromium Hydroxide Green | 0.005 |
| Tocopherol | 0.0002 |
| Water | QS |

Test Methodology:

Two 1 cm diameter test sites were outlined on the volar aspect of each forearm of a subject, for a total of four test sites. Ten microliters of a 1:50 dilution of poison oak/poison ivy oleoresin were applied with disposable micropipettes to 0.6 cm diameter discs of filter paper. The discs were left open to the air for 30 minutes and then carefully applied with forceps to each of the four test sites. The oleoresin-loaded filter paper discs were then covered with occlusive tape (Blenderm, 3M) and left in place for fifteen minutes. After 15 minutes, all of the poison ivy patches were removed and discarded. Sebutapes were applied to one site per forearm of each subject, allowed to remain for 60 seconds and then removed from each of the two sites.

Each of the remaining two test sites was washed with the composition of Example 2 according to the standard instructions. The sites were allowed to air dry for approximately 15 minutes. Sebutapes were then applied to the final two sites, allowed to remain for 60 seconds and then removed.

The Sebutapes were placed in labeled containers and then were extracted in 500 microliters of absolute ethanol. The ethanol samples were then spectrophotometrically analyzed for oleoresins using a Flurorolog Spectrophotometer. The results are shown in Table 1 below.

TABLE 1

| Sample Remaining After Washing | % Oleoresin |
|---|---|
| Control (no washing) | 100 |
| Composition of Example 1 | 5 |
| Composition of Example 1 on a wipe | 6 |

The data above demonstrates that the compositions of the present invention are effective at removing oleoresins from the skin.

| | EXAMPLE 3 |
|---|---|
| | Formula % (w/w) |
| Ethyl alcohol | 75 |
| Isobutylene/Ethylmaleimide/ | 8 |
| Hydroxyethylmaleimide Copolymer | |
| Pramoxine HCL | 1.0 |
| Di-PPG-2 Myreth-10 Adipate | 0.75 |
| Zinc Acetate | 0.12 |
| t-butyl alcohol | 0.08 |
| Water | QS |

Compositions as described in Example 3 may be applied to each site of an individual that has been contacted with the oleoresins and washed with compositions of the present invention as described in Examples 1 and 3. Such application will provide ongoing treatment of skin areas having been contacted with the oleoresins.

We claim:

1. A composition suitable for removal of urushiol from an area of skin having been exposed thereto, comprising:
an alcohol ethoxylate; and
sodium lauryl sarcosinate; said alcohol ethoxylate and said sodium lauryl sarcosinate being present in said composition in relative amounts effective to remove said urushiol from said skin, wherein said composition is essentially free of nonyl phenyl ethoxylates.

2. The composition of claim 1 comprising from about 3 to about 10 weight percent of said alcohol ethoxylate and from about 2 to about 15 weight percent of said sodium lauryl sarcosinate, based on total weight of said composition.

3. The composition of claim 2 wherein said alcohol ethoxylate comprises a C₉-C₁₅ alcohol ethoxylate having HLB of from about 9 to 11.

4. The composition of claim 2 comprising less than about 10 weight percent of said nonyl phenyl ethoxylate.

5. The composition of claim 2 comprising less than 1 weight percent of said nonyl phenyl ethoxylate.

6. The composition of claim 3 comprising about 5 weight percent of said C₁₂ alcohol ethoxylate and about 12 weight percent of said sodium lauryl sarcosinate, said composition being free of said nonyl phenyl ethoxylate.

7. A method for treating allergic dermatitis in an area of skin, said method comprising the steps of:
contacting an area of skin having been exposed to urushiol with a first composition comprising an ethoxylate and sodium lauryl sarcosinate under conditions effective to remove at least a portion of urushiol from said skin, removing said first composition and said portion of urushiol from said skin; and
contacting said exposed area of skin with a second composition comprising a therapeutically effective amount of an active ingredient suitable for treating allergic dermatitis.

8. The method of claim 7 wherein said ethoxylate is selected from the group consisting of a nonyl phenyl ethoxylate and an alcohol ethoxylate.

9. The method of claim 8 wherein said first composition comprises from about 3 to about 40 weight percent of said ethyoxylate and from about 2 to about 20 weight percent of said sodium lauryl sarcosinate, based on the total weight of said composition.

10. The method of claim 9 wherein the weight ratio of said ethoxylate to said sodium lauryl sarcosinate is from about 1:2.5 to about 2.5:1.

11. The method of claim 7 wherein said active ingredient comprises an analgesic.

12. The method of claim 9 wherein said active ingredient comprises an analgesic.

13. The method of claim 7 wherein said ethoxylate comprises a C₉-C₁₅, alcohol ethoxylate having HLB of from about 9 to 11 and wherein said ethoxylate is essentially free of a nonyl phenyl ethoxylate.

14. The method of claim 14 wherein said first composition comprises from about 3 to about 10 weight percent of said C₉-C₁₅ alcohol ethyoxylate and from about 2 to about 15 weight percent of said sodium lauryl sarcosinate, based on total weight of said composition.

15. The method of claim 7 wherein said second composition remains in contact with said skin for a time of from about 1 to about 24 hours.

16. The method of claim 7 wherein said second composition is applied more than once.

17. The method of claim 7 wherein said first composition is applied to said skin by a lotion, spray, gel or wipe.

18. A kit suitable for treatment of allergic dermatitis in an area of skin, comprising:
a first composition comprising an ethoxylate and sodium lauryl sarcosinate in relative amounts effective to remove urushiol from an area of skin having been exposed to said urushiol; and
a second composition comprising a therapeutically effective amount of an active ingredient suitable for treating allergic dermatitis.

19. The kit of claim 18 wherein said ethoxylate is selected from the group consisting of a nonyl phenyl ethoxylate and an alcohol ethoxylate.

20. The kit of claim 18 wherein said active ingredient comprises an analgesic.

21. The kit of claim 7 wherein said ethoxylate comprises a C₉-C₁₅ alcohol ethoxylate having HLB of from about 9 to 11 and wherein said ethoxylate is essentially free of a nonyl phenyl ethoxylate.

22. The kit of claim 21 wherein said active ingredient comprises an analgesic.

23. The kit of claim 18 comprising an article having incorporated therewith said first composition.
